(19) United States
(12) Patent Application Publication
(10) Pub. No.: US 2026/0345342 A1
(43) Pub. Date: Nov. 5, 2026

(54) BONE SCREW AND SYSTEMS FOR BONE FUSION

(71) Applicant: Nordic Health Service Oy, Turku (FI)

(72) Inventor: Janek Frantzen, Turku (FI)

(21) Appl. No.: 19/734,444

(22) Filed: Jul. 8, 2026

Related U.S. Application Data
(63) Continuation of application No. 19/229,351, filed on Jun. 5, 2025, now abandoned.

(30) Foreign Application Priority Data
Jun. 6, 2024 (GB) GB2408032.7

(51) Int. Cl. A61F 2/44; A61F 2/30

(52) U.S. Cl. CPC A61F 2/447; A61F 2/30749; A61F 2002/30421; A61F 2002/30593

(57) ABSTRACT

Disclosed is a bone screw and a fixation system specifically configured for fixing a bone fusion device into a human or animal body with the aid of a specifically designed fastening unit. A bone fusion system including at least two such bone screws in said bone fusion device is also disclosed along with said fastening unit.

### CROSS-REFERENCE TO RELATED APPLICATIONS

This application claims priority to United Kingdom Patent Application No. GB2408032.7 filed Jun. 6, 2024, the disclosure of this application is expressly incorporated herein by reference in its entirety.

### TECHNICAL FIELD

The present disclosure relates generally to medical devices and, more specifically, to bone screws and systems for bone fusions.

### BACKGROUD

Bone fusion is a surgical procedure in which two or more bones, bone structures or bone parts are permanently joined into one solid structure. A need for bone fusion typically arises from damage caused by trauma, disease or degenerative defects. A common type of complex and challenging bone fusion is spinal fusion, often carried out to alleviate chronic pain.

There is a need for improved systems for bone fusion, especially for spinal fusion.

### SUMMARY

In one aspect, an embodiment of the present disclosure provides a bone screw comprising:
an elongated shaft having a straight proximal region and a tapered distal region with one or more threads disposed along the length of the tapered distal region, and one or more helical indentations disposed along the elongated shaft in a direction opposite to the direction of said one or more threads;
a head disposed at the proximal end of the elongated shaft; and
a tip disposed at the distal end of the elongated shaft.

In another aspect, an embodiment of the present disclosure provides a fixation system for a bone fusion device, comprising:
a plate member configured to be coupled to a spacer member in order to form a bone fusion device, the plate member having a central access port extending therethrough, and at least one pair of a first bone screw passage and second bone screw passage in an oblique angle around the access port through the plate member; and
a bone screw according to the present disclosure in at least one of the bone screw passages.

In a yet another aspect, an embodiment of the present disclosure provides a bone fusion system, comprising:
a bone fusion device having an anterior wall, posterior wall, a first lateral wall, a second lateral wall, and a central cavity open to a superior and an inferior surface, wherein
the anterior wall has a central access port extending therethrough, and at least one pair of a first bone screw passage and second bone screw passage in an oblique angle around the access port through the anterior wall,
the posterior wall having a blind-hole at the midpoint of its proximal surface, the blind-hole being configured to detachably receive a distal end of a rod arrangement in a fastening unit specifically configured for fixing the bone fusion device into a human or animal body; and
a bone screw according to the present disclosure in at least one of the bone screw passages.

In a still another aspect, an embodiment of the present disclosure provides a fastening unit for fastening the bone fusion system according to the present disclosure into a human or animal body, comprising:
a drive member comprising:
a rod arrangement having a proximal portion and a distal portion; and
a concavely conical cogwheel stably fixed between said proximal portion and distal portion of the rod arrangement; and
a guide member comprising:
an elongated body having a central aperture extending through the plate member, the central aperture removably received by the proximal portion of the rod arrangement; and
two guidewires, one coupled to each end of the elongated body.

Further aspects, embodiments and details are set forth in following figures, detailed description, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIG. 1A is a side view of a cannulated bone screw with a left-handed thread and a right-handed helical indentation according to an embodiment of the present disclosure.

FIG. 1B is a side view of a cannulated bone screw with a right-handed thread and a left-handed helical indentation shown in FIG. 1A.

FIG. 1C is a cross-sectional side view of a cannulated the bone screw shown in FIG. 1A.

FIG. 2A is a side view of a cannulated bone screw with a left-handed thread and a right-handed helical indentation according to another embodiment of the present disclosure.

FIG. 2B is a side view of a cannulated bone screw with a right-handed thread and a left-handed helical indentation shown in FIG. 2A.

FIG. 2C is a cross-sectional side view of a cannulated the bone screw shown in FIG. 2A.

FIG. 3 is a perspective view of a fixation system according to an embodiment of the present disclosure, including a plate member with two integrated bone screws shown in FIG. 2A.

FIG. 4A is a top view of a bone fusion device according to an embodiment of the present disclosure.

FIG. 4B is a top view of another embodiment of a bone fusion device of the present disclosure, assembled by coupling a plate member to a spacer member.

FIG. 5A shows a perspective view of a bone fusion system comprising a bone fusion device shown in FIG. 4A engaged with two bone screws shown in FIG. 1A. A perspective view and a side view of a cap for sealing a central access port and proximal ends of two bone screw passages is also shown.

FIG. 5B is a perspective view of the bone fusion system shown in FIG. 5A with the access port and proximal ends of the two bone screw passages being sealed with the cap also shown in FIG. 5A.

FIG. 5C is a side view of the bone fusion system shown in FIG. 5A.

FIG. 5D is a front view of the bone fusion system shown in FIG. 5A.

FIG. 5E is a front view of the sealed bone fusion system shown in FIG. 5B.

FIG. 5F is top view of the bone fusion system shown in FIG. 5A.

FIG. 6A is a perspective view of another embodiment of the bone fusion system shown FIG. 5A.

FIG. 6B is a perspective view of the bone fusion system shown in FIG. 6A with a sealed access port and sealed proximal ends of bone screw passages.

FIG. 6C is a side view of the bone fusion system shown in FIG. 6A.

FIG. 7A is a perspective view illustrating engagement of two bone screws with a driver member of a fastening unit specifically configured for fixing a bone fusion device described herein in its desired location in a human or animal body.

FIG. 7B is a top view of the engagement shown in FIG. 7A.

FIG. 7C is a side view of the engagement shown in FIG. 7A.

FIG. 7D is a view along the anterior-posterior axis of the engagement shown in FIG. 7A.

FIG. 8A is a perspective view illustrating engagement of two bone screws with a bone fusion device shown in FIG. 3A and a driver member and guidewires of a fastening unit specifically configured for fixing the bone fusion device in its desired location in a human or animal body.

FIG. 8B is a top view of the engagement shown in FIG. 8A.

FIG. 8C is a side view of the engagement shown in FIG. 8A.

FIG. 8D is a view along the anterior-posterior axis of the engagement shown in FIG. 8A.

FIG. 9A is a perspective view illustrating engagement of two bone screws with a fastening unit described herein at an early phase of a process of fixing a bone fusion device in its desired location in a human or animal body. The bone fusion device to be fixed is not shown for illustrative purposes.

FIG. 9B is a perspective view of the engagement shown in FIG. 9A at a later phase of the process of fixing a bone fusion device in its desired location in the body.

FIG. 9C is a top view of the engagement shown in FIG. 9A.

FIG. 9D is a top view of the engagement shown FIG. 9B.

FIG. 9E is a side view of the engagement shown in FIG. 9A.

FIG. 9F is a side view of the engagement shown FIG. 9B.

FIG. 9G is a view along the anterior-posterior axis of the engagement shown in FIG. 9A.

FIG. 9H is a view along the anterior-posterior axis of the engagement shown FIG. 9B.

FIG. 10A is a perspective view illustrating engagement of two bone screws with bone fusion device shown in FIG. 3A and a fastening unit described herein at an early phase of a process of fixing the bone fusion device in its desired location in a human or animal body.

FIG. 10B is a perspective view of the engagement shown in FIG. 10A at a later phase of the process of fixing the bone fusion device in its desired location in the body.

FIG. 10C is a top view of the engagement shown in FIG. 10A.

FIG. 10D is a top view of the engagement shown FIG. 10B.

FIG. 10E is a side view of the engagement shown in FIG. 10A.

FIG. 10F is a side view of the engagement shown FIG. 10B.

FIG. 10G is a view along the anterior-posterior axis of the engagement shown in FIG. 10A.

FIG. 10H is a view along the anterior-posterior axis of the engagement shown FIG. 10B.

FIGS. 11A-11C are perspective views illustrating an engagement of a fastening unit with a bone fusion device shown in FIG. 3A at successive points in time, in accordance with an embodiment of the present disclosure.

FIG. 12A is a perspective cross-sectional view illustrating the engagement shown in FIG. 11C.

FIG. 12B is a top view of the engagement shown in FIG. 11C.

FIG. 12C is a cross-sectional view of the engagement shown in FIG. 13B.

FIGS. 13A-13C are perspective views illustrating an engagement of a fastening unit with a bone fusion device shown in FIG. 3B at successive points in time, in accordance with an embodiment of the present disclosure.

FIG. 14A is a perspective cross-sectional view illustrating the engagement shown in FIG. 13C.

FIG. 14B is a top view of the engagement shown in FIG. 13C.

FIG. 14C is a cross-sectional view of the engagement shown in FIG. 13C.

### DETAILED DESCRIPTION

The present disclosure relates to means for the management of conditions that benefit from bone fusion.

It is to be understood that the terminology used herein is for the purpose of describing particular embodiments only and is not intended to limit the scope of the present invention, which will be limited only by the appended claims.

As used herein, the singular expressions “a”, “an” and “the” mean one or more. Thus, a singular noun, unless otherwise specified, carries also the meaning of the corresponding plural noun.

The terms “one or more” and “at least one” are used herein interchangeably, and can refer to exactly one or to multiple, such as two, three, four, five, six, seven, eight or even more. The term "a plurality of' refers to two or more.

As used herein, the terms "first", "second" and the like are merely for the descriptive purpose but cannot be understood as indicating or implying a relative importance.

As used herein, the term “and/or” in a phase such as “X and/or Y” means either “X and Y” or “X or Y” and shall be taken to provide explicit support for both meanings or for either meaning.

The terms “comprising”, “including” and “having” are used herein interchangeably, and are intended to be construed in a non-exclusive manner, i.e. allowing for features not explicitly described also to be present.

### Bone screws

In one aspect, the disclosure provides a bone screw, especially a bone screw for use in securing a bone fusion device, such as an interbody fusion implant, in its intended place in a human or animal body.

The bone screw includes a screw head, a shaft and a tip. In some preferred embodiments, the tip is a self-drilling screw tip.

The shaft is provided with a proximal region adjacent to the screw head, the rest of the shaft including a tapered distal region.

The shaft is also provided with at least one thread, more specifically at least one external thread (i.e. a projecting helical ridge of a screw), which is disposed at least along the distal region such that it covers either the whole distal region or at least a distal portion thereof (i.e. a portion next to the screw tip). In some embodiments, said at least one thread may continue also along the proximal region such that it covers the whole proximal region or a distal portion thereof (i.e. a portion next to the distal region). Thus, in different embodiments, the threaded region may encompass either a distal portion of the distal region, the whole distal region, the whole distal region and a distal portion of the proximal region, or the whole distal portion and the whole proximal portion, i.e. the whole shaft. In an embodiment, the proximal region is non-threaded, whereas the distal region is threaded.

In some embodiments, the distal region is tapered at an angle that is dimensioned to match with a cogwheel of a fastening unit specifically designed for fixing a bone fusion device described herein in its desired location in a human or animal body. At least the distal region is configured to be screwed into a human or animal bone.

The proximal region, also called a shank, is straight, i.e., non-tapered, and it may be either threaded or non-threaded in accordance with what is stated above. Without limitation to any theory, if the proximal region is non-threaded, it may facilitate detaching of a fastening unit described hereinbelow once the screw has been inserted into a human or animal body. Furthermore, again without limitation to any theory, if the proximal region is straight, it may contribute to pulling bones, bone structures or bone parts to be fused towards a bone fusion device described herein, when said device is being fixed with said bone screw.

The thread may be either left-handed or right-handed, as desired. However, for use in a bone fusion system provided herein, bone screws with the same handedness should be used, be it a left- handed or right-handed thread.

In some embodiments, the shaft is provided with a single thread, which may have a constant pitch or a variable pitch extending along the threaded region. In some other embodiments, the threaded region is provided with a plurality of parallel threads extending helically around the shaft, the threaded region thus being multi-threaded, such as double-threaded. In some further embodiments, the threaded region may be provided with two or more differently threaded portions. For example, in some embodiments, the threaded region may have a proximal portion and a distal portion, wherein a first thread extends across both the proximal portion and the distal portion, wherein a second thread extends across the proximal portion only and is located between adjacent windings of the first thread. In other words, the threaded region may have a single-threaded distal portion and a double-threaded proximal portion.

The shaft is also provided with at least one helical indentation in a direction opposite to the thread. Thus, if the thread is right-handed, the at least one helical indentation is left-handed, and vice versa.

The at least one helical indentation makes the thread helically discontinuous in a direction opposite to the thread. In an embodiment, the depth of the helical indentation is such that at the threaded region it only cuts the thread making the thread helically discontinuous but does not extend into the screw core, i.e. the screw base having the least diameter of the screw. In another embodiment, the depth of the helical indentation is such that at the threaded region it not only cuts the thread but also extends into the screw core.

In an embodiment, the at least one helical indentation extends along the whole length of the shaft, thus encompassing the threaded region and, if present, also the non-threaded region. In other words, the helical indentation(s) may extend across the proximal region and the distal region, regardless of whether the shaft includes a non-threaded region or not. If a non-threaded region is present, the depth of the helical indentation(s) is such that it extends into the shaft core at least across the non-threaded region. In a further embodiment, the helical indentation(s) may also extend across the screw head.

In some embodiments, the shaft is provided with a single helical indentation. In such cases, the root-to-root pitch is configured to match with the dimensions of a cogwheel of a fastening unit described hereinbelow such that rotation of the cogwheel drives the screw forward pass the cogwheel and eventually into a human or animal bone.

In some other embodiments, the shaft may be provided with a plurality of adjacent indentations extending helically around the shaft. In such cases, the distance between the roots of the adjacent indentations is configured to match with the dimensions of a cogwheel of a fastening unit mentioned above. Also in these cases, rotation of the cogwheel drives the screw forward pass the cogwheel and eventually into a human or animal bone.

In some embodiments, the diameter of the screw head is larger than the diameter of the proximal end (i.e. the shank) of the shaft adjacent to the screw head. In some embodiments, such a screw head is dimensioned to be larger than a bone screw passage of a bone fusion device disclosed herein, thereby allowing the bone screw to secure the bone fusion device to a human or animal bone.

The diameter of the shank is usually constant and equal to the diameter of the proximal end of the tapering distal region. In other words, except for the proximal end of the tapering region, the diameter of the shank is larger than the diameter of the tapering region. Therefore, securing of a bone fusion device to its intended place in a human or animal body can, in some embodiments, be achieved even without a screw head wider than the proximal end of the shaft adjacent to the screw head. In such instances, the screw head has a diameter that equals the proximal diameter of the shaft adjacent to the screw head, said diameter being dimensioned to be larger than a bone screw passage of a bone fusion device, thereby allowing the bone screw to secure said device to a receiving human or animal bone.

As a feature independent from the diameter of the screw head, the screw head may in some embodiments be provided with a tooling recess for engaging a fastening tool, such as a screwdriver, an Allen wrench, or the like, to enable removal of the bone screw, if needed. For example, the head of the bone screw may have a Torx™ recess configured to be turned with a screwdriver or other fastening tool having a corresponding Torx™ head. Alternatively, the tooling recess may be configured to be turned with, for example, a Phillips™ screwdriver, a Pozidriv™ screwdriver, a Supadriv™ screwdriver, or the like.

In some embodiments, the bone screw is fully cannulated, i.e. provided with a longitudinal through-bore or cannula extending through the whole screw length including the head, the shaft and the tip. This feature applies especially to short bone screws, but is by no means limited thereto. In other words, a bone screw of any length may be fully cannulated.

However, in some other embodiments, the bone screw need not be fully cannulated, i.e. the longitudinal bore need not extend through the whole shaft and/or the tip of the bone screw. In other words, it is enough that the bone screw is cannulated through the screw head and at least a proximal part of the shaft, including the proximal region and at least a portion of the distal region adjacent to the proximal region. Such bone screws can be denoted as partly cannulated bone screws. This embodiment applies especially to long bone screws. Those skilled in the art can easily select a bone screw with a longitudinal bore having a length that is appropriate for the intended purpose.

For the sake of simplicity, the term “cannulated” bone screw, as used herein, encompasses both “at least partly cannulated” and “fully cannulated” bone screws, unless the context clearly indicates otherwise.

The longitudinal bore (i.e. cannula) is configured to receive a guidewire of a fastening unit tool described hereinbelow, thereby facilitating better screw alignment and insertion of the bone screw into a receiving bone with the aid of the fastening unit. After the insertion, the guidewire along with the rest of the fastening unit is removed and the bone screw is left in its place in the human or animal bone. In some other embodiments, the guidewire may be left in the bore whereas the rest of the fastening unit is removed after the bone fusion device has been fixed.

Dimensions of the bone screw, including its length, core diameter (the root-to-root diameter), outside diameter (the thread-to-thread dimeter), dimensions of the longitudinal bore, as well as the size and the shape of the head and the tip, may vary depending on various variables, such as the size and anatomy of a patient to be treated and/or the type and extent of the bone fusion to be achieved.

In some embodiments, the screw shaft may be perforated to include one or more through-holes extending from the cannulated hollow interior through a lateral surface of the screw shaft. The through-holes may be scattered along the whole length of the screw shaft, as desired, either evenly or unevenly. If after removal of a guidewire of a fastening unit from the cannula, an agent of interest is administered into the cannula, the through-holes will deliver the agent of interest to a receiving bone area.

Non-limiting examples of agents of interest to be delivered to the receiving bone area via the one or more through-holes described above include bone cement, bone stimulating agents such as growth factors, including bone morphogenic proteins and other bone growth promoting growth factors, cells such as osteoblasts and pluripotent or multipotent stem cells, and medicaments such as antibiotics.

The bone screw may be prepared from any biocompatible material suitable for insertion into bone. Non-limiting examples of such materials include metals and metal alloys, such as titanium and titanium alloys, composites, fibre-reinforced composites, such as those reinforced with e.g. carbon fibres or glass fibres, and any combinations thereof.

In some embodiments, the bone screw may be coated, suitable coatings including, but not being limited to, hydroxyapatite, titanium oxide or any other metals and composites. In some other embodiments, the bone screw may be uncoated.

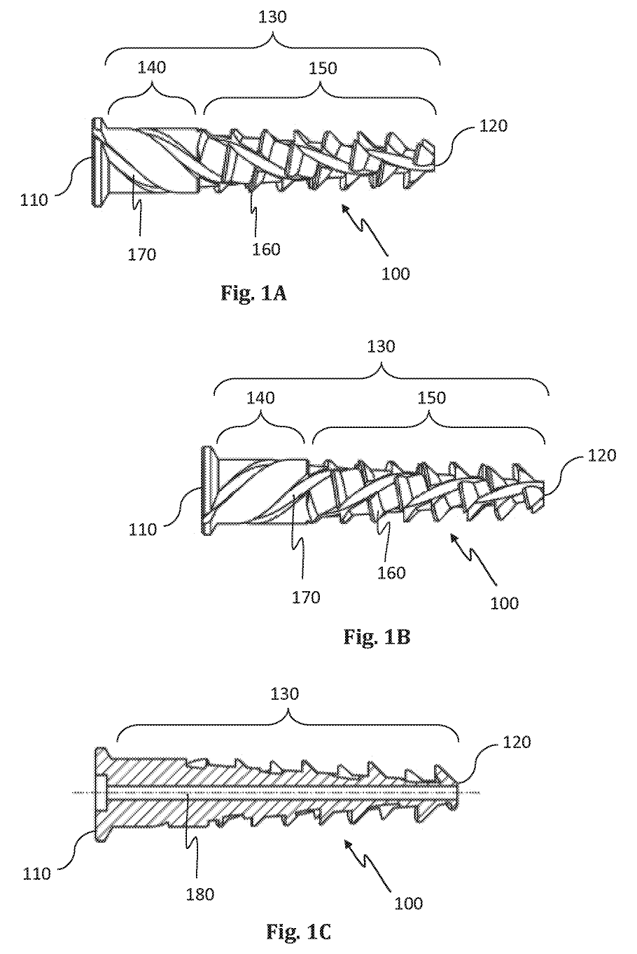

Turning now to the drawings, FIGS. 1A and 1B illustrate bone screws according to some embodiments of the present disclosure. The bone screw 100 includes a head 110, a tip 120, and an elongated shaft 130. The shaft 130 has a straight proximal region 140 which is non-threaded, and a tapered distal region 150 having one or more parallel threads 160. The shaft 130 has one or more parallel helical indentations 170 in a direction opposite to the one or more threads 160, rendering the one or more threads 160 helically discontinuous. The bone screw 100 includes a longitudinal bore 180 which may in some embodiments extend along the whole screw length including the head 110, the shaft 130 and the tip 120 as is illustrated in FIG. 1C.

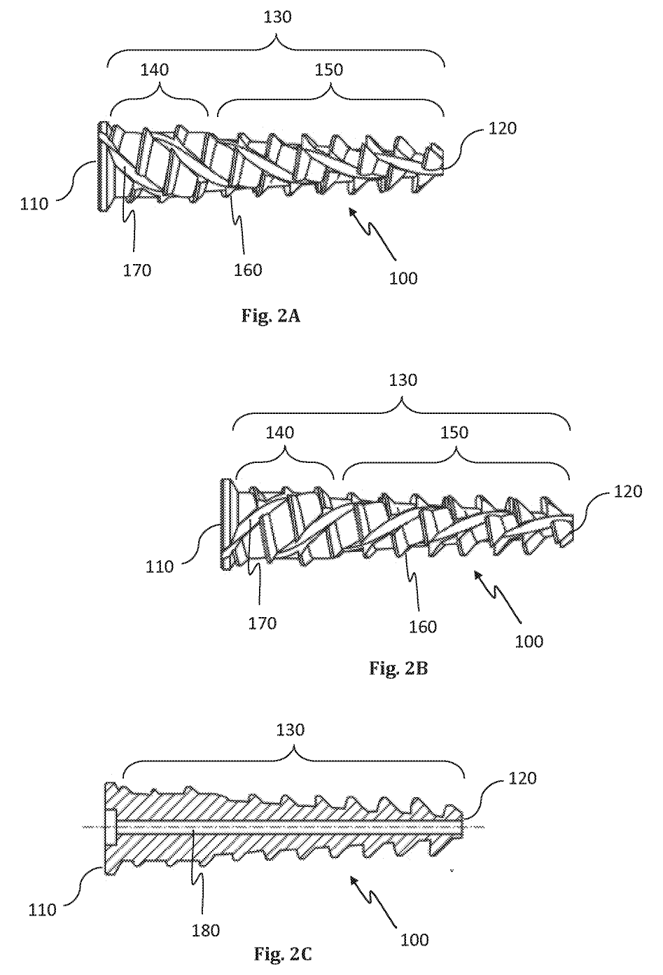

FIGS. 2A and 2B illustrate bone screws according to some other embodiments of the present disclosure. The bone screw 100 includes a head 110, a tip 120, and an elongated shaft 130. The shaft 130 has a straight proximal region 140 and a tapered distal region 150. One or more parallel threads 160 are disposed along the shaft 130 such that the straight proximal region 140 and the tapered distal region 150 are encompassed. The shaft 130 has one or more parallel helical indentations 170 in a direction opposite to the one or more threads 160, rendering the one or more threads 160 helically discontinuous. The bone screw 100 includes a longitudinal bore 180 which may in some embodiments extend along the whole screw length including the head 110, the shaft 130 and the tip 120 as is illustrated in FIG. 2C.

### Bone fusion system and related aspects

In one aspect, the present disclosure provides a fixation system for a bone fusion device, i.e. for a medical device configured to be inserted between two or more distinct bones or bone structures, or between two or more parts of a single bone, for the purpose of permanently joining the bones, bone structures or bone parts together. The bone fusion device is suitable for various medical applications, such as for use in spinal fusion as well as for fusing joints and any other bones or bone structures. The bone fusion device may also be used in an area of missing bone, thereby joining and mediating fusion of the surrounding bone areas. For use in spinal fusion, the bone fusion device may be denoted as an interbody fusion implant, i.e., an interbody fusion cage.

The fixation system comprises a plate member and at least two integrated bone screws described hereinabove, wherein the plate member is configured to be coupled to a spacer member. When coupled, the plate member and the spacer member form a bone fusion device.

The plate member includes an anterior wall and a first lateral extension projecting backwards from a first end of the anterior wall and a second lateral extension projecting backwards from a second end of the anterior wall. Each of the first and the second lateral extensions has an attachment portion configured to be received by a spacer member, more specifically by a corresponding attachment portion in a corresponding lateral wall of the spacer member.

The plate member has at least two bone screw passages, i.e., canals, extending through its anterior wall at an oblique angle, configured to receive a bone screw disclosed herein and to define a bone screw axis.

In some embodiments, the plate member may have more than two bone screw passages, such as four, six or eight bone screw passages extending through the anterior wall. Having an even number of bone screw passages enables the fusion device be secured in its place in a human or animal body by use of a fastening unit described hereinbelow, which unit is configured to fasten two bone screws at a time. In some embodiments, the plate member has two bone screw passages. To put it differently, the plate member may have one or more pairs of bone screw passages.

The bone screw passages are positioned, i.e. disposed, symmetrically around a bore, denoted herein as an access port, at the midpoint of the anterior wall of the plate member. For each pair of bone screw passages, a first bone screw passage is angled through the anterior wall in an oblique upward direction and a second bone screw passage is angled through the anterior wall in an oblique downward direction.

In some embodiments, especially in those relating to interbody fusion implants, each bone screw passage in a pair of first and second bone screw passages, while being angled in either oblique upward or downward direction, is also angled in an inward direction, i.e., towards the vertical midline of the plate member along the anterior-posterior axis. In other words, the inner opening of the bone screw passage at the posterior surface of the anterior wall is closer to the midpoint of the anterior wall than the outer opening of the bone screw passage at the anterior surface of the anterior wall.

In some embodiments, the opening of the first bone screw passage at the posterior surface of the anterior wall is through an upper inner corner between the anterior wall and the first lateral extension (i.e., between the anterior wall and the first lateral wall in an assembled bone fusion device), whereas the opening of the second bone screw passage at the posterior surface of the anterior wall is through a lower inner corner between the anterior wall and the second lateral extension (i.e., between the anterior wall and the second lateral wall in an assembled bone fusion device).

In some embodiments, the plate member may include one or more bone screw passages in addition to the at least one pair of first and second bone screw passages described above. The one or more additional bone screw passages, if present, need neither be deposited symmetrically around the access port nor be in an oblique angle. Moreover, the one or more additional bone screw passages are not limited to those configured to receive bone screws disclosed herein. Accordingly, in some embodiments, the plate member may include, for example, three bone screw passages, namely one pair of a first and second bone screw passages disclosed above and a further bone screw passage.

The bone screw passages are configured to be sufficiently large to allow a bone screw to pass therethrough such that its distal end is insertable into a receiving bone structure but not large enough to allow a screw head of the bone screw through the bone screw passage. This allows the bone screw to secure the bone fusion device to its desired location in a human or animal body.

The bone screw passage may be countersunk or recessed at its anterior end to allow the screw head to be embedded into a portion of the anterior wall. Consequently, the screw head will reside below the anterior surface of the anterior wall.

In some embodiments, the anterior opening of the bone screw passages may be equipped with a locking wire extending across said opening, the locking wire being configured to give way when a bone screw is being inserted and to return to its position across the bone screw opening once the bone screw has been inserted, thereby preventing backout of the bone screw. Nickel-titanium shape memory metal Nitinol is a non-limiting example of a material suitable for use as a locking wire.

The plate member has a bore (i.e., an access port) disposed at its midpoint, extending through the anterior wall along the horizontal anterior-posterior axis of the plate member. The access port is configured to detachably engage with a fastening unit described hereinbelow for simultaneous insertion of two bone screws placed in their respective bone screw passages in a bone fusion device into their target bone structures, thereby securing the bone fusion device in its intended place in a human or animal body. The anterior end of the access port may be recessed to removably capture a distal portion of a cogwheel of the fastening unit, thereby facilitating fixation of the bone fusion device by keeping the fastening unit in its intended position during fixation. Further details and uses of the access port are discussed later in this description.

Spacer members to be coupled with the plate members are not limited. Generally, the spacer member comprises a posterior wall and a first lateral wall projecting frontwards from a first end of the posterior wall and a second lateral wall projecting frontwards from a second end of the posterior wall. Each lateral wall has an attachment portion configured to be received by a plate member, more specifically configured to be coupled to a corresponding attachment portion in a lateral extension of the plate member. Thus, the coupled first lateral extension of the plate member and the first lateral wall of the spacer member from a first lateral wall of an assembled bone fusion device, whereas the coupled second lateral extension of the plate member and the second lateral wall of the spacer member form a second lateral wall of the assembled bone fusion device.

The spacer member may have any desired design. In some embodiments, the spacer member may have a solid or fenestrated posterior wall and/or solid or fenestrated lateral walls. In some further embodiments the spacer may have a truss structure.

The space member is sized and shaped to fit between bones or bone structures to be fused. Preferably, the spacer member has an anatomical shape, meaning that at least one of its superior and the inferior surfaces has a curvature that substantially follows the curvature of the bone structure it is to be interfaced with. In some embodiments, the spacer member may have an anterior height that is substantially equal to the posterior height. However, in some other embodiments, the spacer member may have an anterior height that is different from its posterior height. To this end, at least one of the superior surface and the inferior surface may be at least partially tapering or convex with respect to the horizontal midline of the spacer member at a predetermined angle along the anterior-posterior axis. Generally, the superior surface and/or the inferior surface may be configured to have an angle ranging from zero to about 20° with respect to the horizontal midline of the spacer member along the anterior-posterior axis, depending on the embodiment in question and the intended use of the bone fusion device assembled from the plate member and the spacer member.

In some embodiments, at least one of the superior surface and the inferior surface may have serrations, teeth or the like to create a rough surface configured to diminish or prevent movement of the bone fusion device after surgery.

Regardless of the design of the spacer member, its posterior wall should have, at the midpoint of its anterior surface, a blind hole configured for a detachable engagement with a fastening unit described hereinbelow, for facilitating proper alignment of the fastening unit. In some embodiments, the blind-hole contains another, narrower blind-hole at the midpoint of the first blind-hole configured to receive a fastening stick of a fastening unit described later in this description. The inner blind-hole has an internal thread configured for detachable engagement with an external thread at the distal end of the fastening stick.

In some embodiments, the plate member and the spacer member are preassembled to form a bone fusion device described herein. The manner by which the plate member and the spacer member are configured to be attached together to form an assembled bone fusion device is not limited.

In some other embodiments, the bone fusion device is manufactured as a single-piece object having a walled perimeter formed of an anterior wall, a posterior wall, a first lateral wall and a second lateral wall.

For the sake of simplicity, the following description of the bone fusion device applies regardless of whether it is manufactured as a single object or be assembled from a plate member and a spacer member as described above. It is also to be understood that any above-described features of the plate member, the spacer member and/or a bone fusion device assembled from the plate member and the spacer member apply to a bone fusion device manufactured as a single object even if said features are not repeated when describing such a single-piece bone fusion device.

The walls of the bone fusion device create a central cavity that is substantially open to the superior (top) and inferior (bottom) surfaces of the bone fusion device. The superior surface is configured to interface with a first bone structure, while the inferior surface is configured to interface with a second bone structure, the intervening bone fusion device mediating fusion of the first and the second bones or bone structures.

If the fusion device is an interbody fusion implant, it is sized to be inserted into an intervertebral space such that the superior surface contacts a first vertebral member and the inferior surface contacts a second vertebral member. In some embodiments, the interbody fusion implant may have an anterior height that is different from its posterior height. Preferably, the height of the anterior wall is greater than the height of the posterior wall, especially if the interbody fusion implant is configured for implantation via an anterior approach. In some further embodiments, the bone fusion device has an anatomical shape such that the superior surface has at least partially tapering or convex curvature that follows the curvature of the end plate of the first vertebral member. Generally, the superior surface may be configured to have an angle ranging from zero to about 20° with respect to the horizontal midline of the bone fusion device along the anterior-posterior axis, as desired.

In some embodiments, at least one of the superior surface and the inferior surface may have serrations, teeth or the like to create a rough surface configured to diminish or prevent movement of the bone fusion device after surgery.

In accordance with what is stated above, the anterior wall of the bone fusion device has at least two bone screw passages extending therethrough, configured to receive a bone screw disclosed herein for insertion into a bone using a fastening unit disclosed herein, thereby securing the bone fusion device between two bones or bone structures.

A first of the at least two bone screw passages is formed through the anterior wall at an oblique angle towards the centre of the superior surface such that when occupied by a first bone screw, the distal portion of the first bone screw is operable to extend beyond the superior surface into a first bone or bone structure immediately facing the superior surface of the central body. Correspondingly, a second of the at least two bone screw passages is formed through the anterior wall at an oblique angle towards the centre of the inferior surface such that when occupied by a second bone screw, the distal portion of the second bone screw is operable to extend beyond the inferior surface into a second bone structure immediately facing the inferior surface of the central body. Consequently, the first and the second bone screws will affix the bone fusion device between the two bones or bone structures. In some embodiments concerning an interbody fusion implant, the bone screws in their respective bone screw passages are operable such that their distal portions extend into the vertebral members immediately above and below the implanted interbody fusion implant to affix the implant within the vertebral space between such vertebral members.

At least in those embodiments which concern interbody fusion implants as the fusion device, it is to be understood that the length of the bone screws to be received in the bone screw passages should be such that their tips do no extend beyond the inner perimeter of the posterior wall. This is to prevent the screws from damaging the vertebral foramen.

In some embodiments, the bone screws have been pre-installed in the bone screw passages of the bone fusion device.

In some embodiments, once the bone fusion device has been fixed into a human or animal body, the access port may be used for filling the central cavity of the bone fusion device with an agent of interest. Once the filling is completed, the access port may be sealed with a cap configured for this purpose, thereby sealing the central cavity. If desired, also the anterior ends of the bone screw passages may be sealed, either with the same cap as the access port or with separate caps. Sealing of the bone screw passages may be beneficial especially in those embodiments, where the bone screws employed include through-holes described above and also the longitudinal bores of the bone screws are filled with an agent of interest after insertion. In some embodiments, the central cavity may be pre-filled with and hold an agent of interest prior to implantation into a human or animal body.

Suitable agents of interest for filling the central cavity include, but are not limited to, any natural or artificial osteoinductive, osteoconductive, osteogenic, or other fusion enhancing material. Some examples of such materials include autograft bone and bone substitutes such as hydroxyapatite and hydroxyapatite tricalcium phosphate bone morphogenic proteins and other bone growth promoting growth factors; cells such as osteoblasts and pluripotent or multipotent stem cells. Said agent may be used with or without any appropriate carrier materials. The agent for filling the central cavity (if to be used at all) may be selected independently from the agent selected for filling the some or all of the longitudinal bores of the bone screws (if to be used at all).

The bone fusion device, including the plate member and the spacer member, should be made of biocompatible material appropriate for human or animal implantation. Non-limiting examples include metals and metal alloys, such as stainless steel, titanium and titanium alloys; polyether ether ketone (PEEK) and other polymers; ceramics; composites and fibre-reinforced composites, such as those reinforced with e.g. carbon fibres or glass fibres; and any combinations thereof. If the bone fusion device is to be assembled from the plate member and the spacer member, they can be made of either the same or different materials. In some embodiments, the plate member is made of a metal or a metal alloy, preferably titanium or titanium alloy, whereas the spacer member is made of a polymer, preferably PEEK.

In some embodiments, the plate member, the spacer member or the whole bone fusion device is coated. Suitable coatings include, but are not limited to, hydroxyapatite, titanium oxide or any other metals and composites.

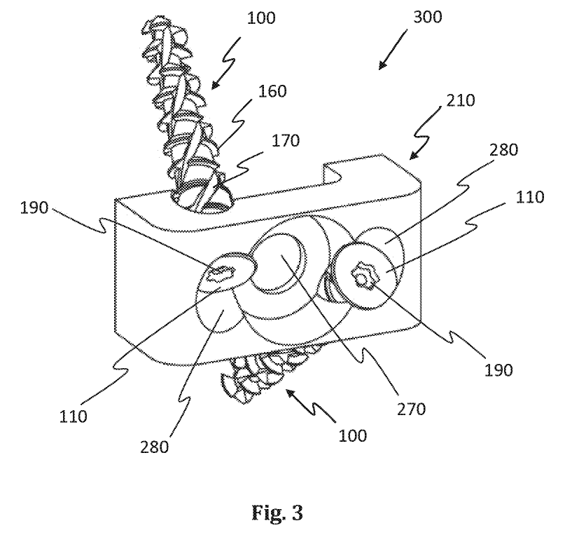

Turning now to the drawings, FIG. 3 illustrates an embodiment of a bone fixation system 300 including a plate member 210 having a recessed central access port 270 extending through the plate member 210, a recessed bone screw passage 280 on both lateral sides of the access port 270, and a bone screw 100 in each bone screw passage 280. The bone screws 100 shown are according to an embodiment, wherein they include a screw head 110 with a tooling recess 190, and a thread 160 and a helical indentation 170 extending along the whole length of the screw shaft 130 in opposite directions.

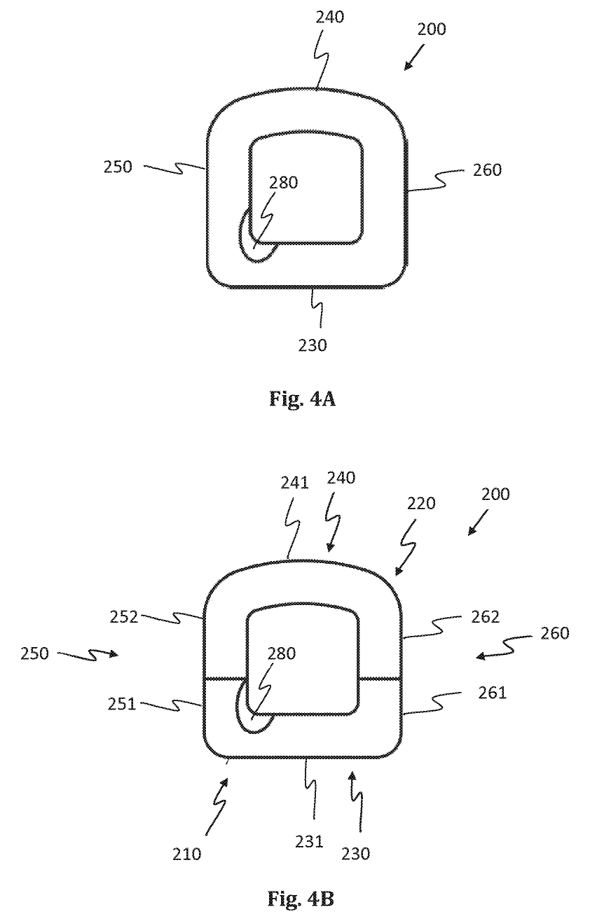

FIGS. 4A and 4B illustrate different embodiments of a bone fusion device 200. FIG. 4A shows an embodiment of the bone fusion device 200 manufactured as a single-piece object, whereas FIG. 4B shows an embodiment of the bone fusion device 200 assembled from a plate member 210 and a spacer member 220. In both cases, the bone fusion device 200 has an anterior wall 230, a posterior wall 240, a first lateral wall 250 and a second lateral wall 260. The plate member 210 includes an anterior wall 231, a first lateral extension 251 and a second lateral extension 261. The spacer member 220 includes a posterior wall 241 and a first lateral wall 252, and a second lateral wall 262. As illustrated in FIG. 4B, the first lateral wall 250 of the bone fusion device 200 may be formed of the first lateral extension 251 of the plate member 210 and the first lateral wall 252 of the spacer member 220. Correspondingly, the second lateral wall 260 of the bone fusion device 200 may be formed of the second lateral extension 261 of the plate member 210 and the second lateral wall 262 of the spacer member 220. Also shown is a posterior end of a bone screw passage 280 located in an upper inner corner between the anterior wall 230 and the first lateral wall 250.

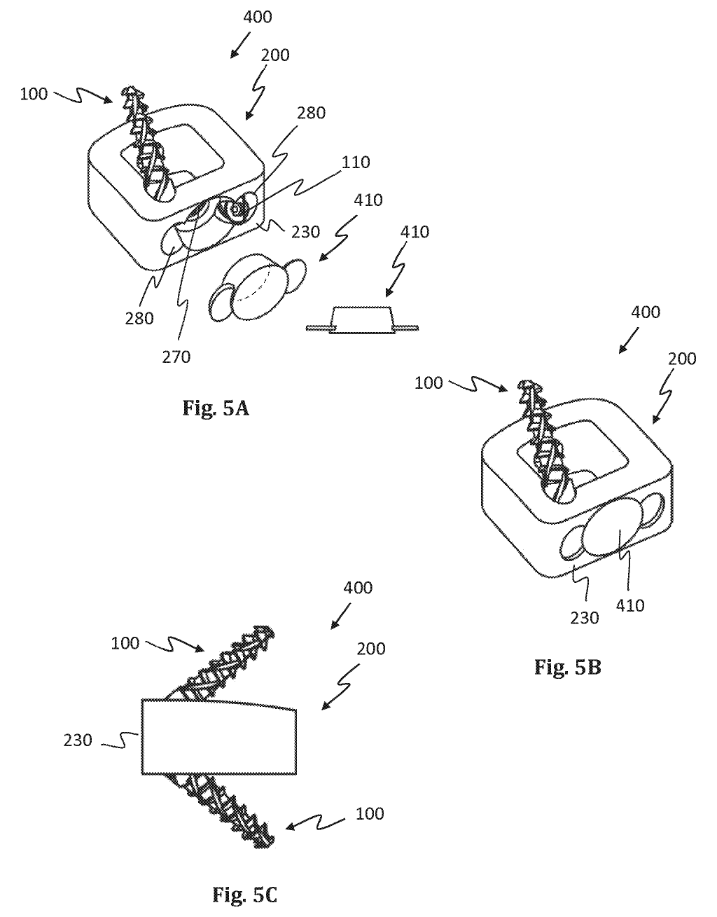

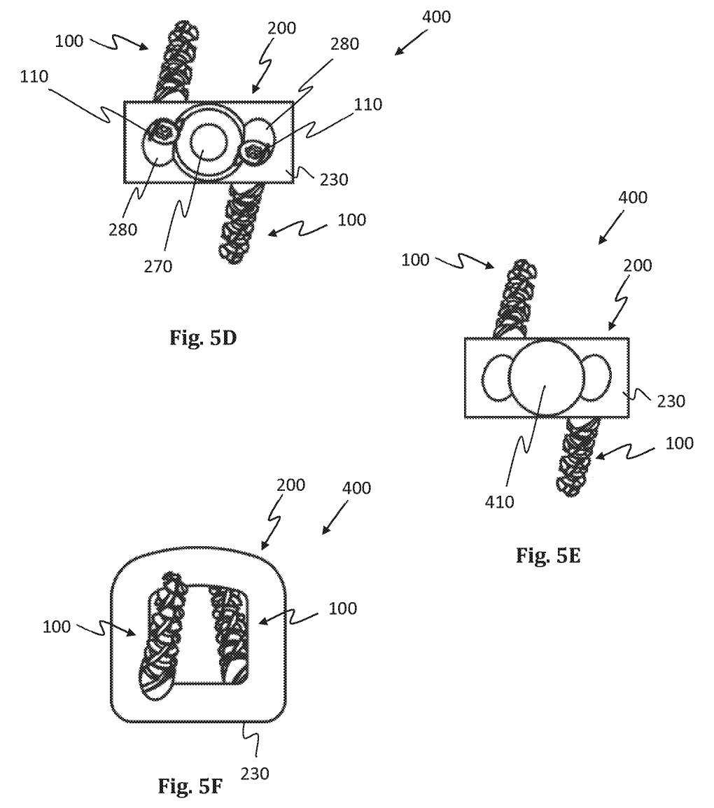

FIGS. 5A-5F show different views of an embodiment of a bone fusion system 400, wherein the bone fusion device 200 is a single-piece object having a central access port 270 and two bone screw passages 280, each passage provided with a bone screw 100. Proximal ends of the bone screw passages 280 may be recessed such that heads 110 of the bone screws 100 will become embedded in the anterior wall 230 of the bone fusion device 200. Also the proximal end on the access port 270 may be recessed. The proximal ends of the bone screw passages 280 and the access port 270 may be sealed with a cap 410 as illustrated in FIGS. 5B and 5E.

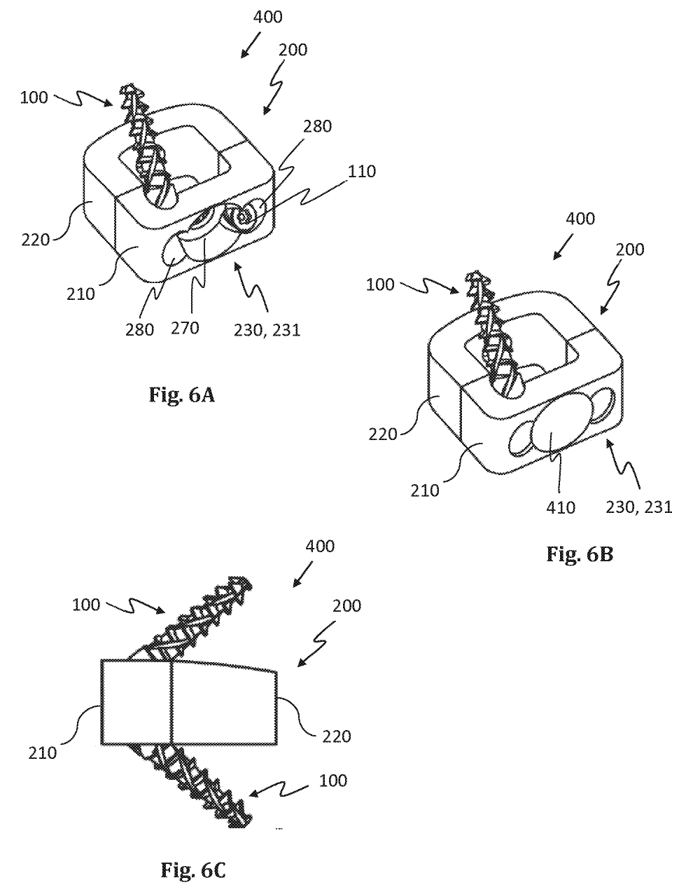

FIGS. 6A-6C show different views of an embodiment of a bone fusion system 400, wherein the bone fusion device 200 is assembled from a plate member 210 and a spacer member 220. The bone fusion device 200 has a recessed central access port 270 and two recessed bone screw passages 280, each passage provided with a bone screw 100. Heads 110 of the bone screws 100 are embedded in the anterior wall 230, 231 of the bone fusion device 200. Also the proximal end on the access port 270 may be recessed. The proximal ends of the bone screw passages 280 and the access port 270 may be sealed with a cap 410 as illustrated in FIG. 6B.

### Fastening unit

In a further aspect, the present disclosure provides a fastening unit for use in inserting bone screws disclosed herein to secure a bone fusion device also disclosed herein in its intended place in a human or animal body.

The fastening unit includes a drive member and a guide member. The drive member is configured to controllably and removably attach the fastening unit to the bone fusion device and to drive the bone screws into the receiving bone while the fastening unit is attached to the bone fusion device. The guide member, in turn, is configured to align and keep the bone screws in an appropriate position with respect to the drive member and the bone fusion device during the process of securing the bone fusion device to a human or animal bone. Once secured, the fastening unit is to be detached and removed.

The drive member includes a rod arrangement and a concavely conical cogwheel stably fixed between a proximal elongated portion and a distal elongated portion of the rod arrangement. To put it differently, the cogwheel has a proximal end and a distal end, the proximal portion of the rod arrangement protruding from the centre of the proximal end of the cogwheel and the distal portion of the rod arrangement protruding from the centre of the distal end of the cogwheel. The drive member can be constructed in different ways. In some embodiments, the proximal and the distal portions of the rod arrangement are provided as distinct proximal and distal elongated rods such that the cogwheel is stably fixed from the midpoints of its proximal and distal ends between the proximal elongated rod and the distal elongated rod, respectively. In some other embodiments the rod arrangement is provided as a single elongated rod configured and stably fixed through the cogwheel along its central axis such that a proximal portion of the elongated rod forms protrudes from the centre of the proximal end of the cogwheel and a distal portion of the rod protrudes from the centre of the distal end of the cogwheel. In some further embodiments, the drive member is manufactured as a single-piece object using techniques readily available in the art. Regardless of how the rod arrangement is being constructed, the proximal and the distal portions thereof may have the same or different diameter. Preferably, at least the distal portion is cylindrical. In some embodiments, the proximal portion may include a handle to improve grip and to facilitate rotating the said proximal portion of the rod arrangement. The cogwheel being stably fixed to the rod arrangement means that rotating the proximal portion of the rod arrangement rotates the whole drive member.

The distal portion of the rod arrangement is dimensioned to fit through the access port of the bone fusion device disclosed herein and to be received by a blind hole at the inner surface (i.e. the proximal surface) of the posterior wall of the device.

In some embodiments, a longitudinal bore extends through the drive member, i.e., through the rod arrangement and the cogwheel, the longitudinal bore being configured to receive a fastening stick having an external thread at its distal end. Said distal end is configured to be received by and screwed into an inner blind-hole of a bone fusion device having a corresponding inner thread. The fastening stick is not attached to the inner surface of the longitudinal bore, thereby allowing the fastening stick and the rest of the drive member be rotated along their longitudinal axes independently from each other. Thus, attaching the fastening stick into the inner blind-hole of the bone fusion device through their matching threads by screwing prevents the fastening unit from slipping during operation, but does not prevent the rest of the drive member be rotated thereby driving the bone screws into the receiving bone. Moreover, fastening of the fastening unit to the bone fusion device enables adjusting the position of the bone fusion device in the body before being fixed to a certain position by the bone screws. For example, if the bone fusion device has been pushed too far into the body, it can be pulled back by pulling the fastening unit that has been fastened to the bone fusion device through the fastening stick. Once the bone screws have been inserted, the fastening unit can be removed by first detaching the fastening stick from the inner blind-hole and then by pulling the fastening unit out from the bone fastening device.

Being concavely conical means that the lateral sides of the cogwheel are not straight but concave such that the proximal end of the cogwheel has a diameter that is larger than the diameter of the distal end, the cogwheel thus having a tapering overall shape (i.e. an imaginary line drawn from a point at the outer rim of the proximal end to a corresponding point at the outer rim of the distal end is tapering). The exact shape and dimensions of the cogwheel may vary, contributing to the angle at which the drive member drives the bone screws. Therefore, the cogwheel is to be shaped and dimensioned such that the desired screw angle is achieved. Also the bone screw passages in the bone fusion device are to be configured to match the desired screw angle. The cogwheel is also to be shaped and dimensioned such that its distal portion is receivable and removably capturable by the access port of the bone fusion device to be implanted.

The cogwheel has radially projecting teeth that are configured to mesh with the one or more helical indentations in the bone screws such that rotation of the cogwheel (through rotation turning of the proximal part of the rod arrangement in the drive member) drives the screws forward pass the cogwheel. In some embodiments, the teeth are aligned in a straight line that intersects at the central axis of the cogwheel when imaginarily extended beyond its distal end.

In some embodiments, the cogwheel may include a helical indentation extending from its proximal end to its distal end, said indentation configured to allow a bone screw having a head with a diameter wider than the shaft adjacent to the head to pass the cogwheel during the process of fastening the bone fusion device into a human or animal body.

The guide member of the fastening unit includes an elongated body, such as a substantially flat plate, having a first end coupled to a first guidewire configured to be received in a longitudinal bore of a first bone screw, and a second end coupled to a second guidewire configured to be received in a longitudinal bore of a second bone screw. The elongated body also has a central aperture between the first and the second end, configured to receive the proximal elongated rod of the drive member therethrough such that the guidewires protrude from the ends of the elongated body towards the distal elongated rod of the drive member.

The guidewires may be coupled to the ends of the elongated body by any appropriate means known to those skilled in the art. The first guidewire is disposed at an angle defined by the cogwheel as explained above, the angle being also substantially parallel to the central axis of the first bone screw passage in the bone fusion device to be implanted. Correspondingly, also the second guidewire is disposed at an angle defined by the cogwheel, the angle being also substantially parallel to the central axis of the second bone screw passage in the bone fusion device to be implanted. As already indicated, each guidewire is configured to be inserted into the longitudinal bore of a bone screw to guide the bone screw to such a position that a helical indentation of the bone screw interacts with a tooth of the cogwheel. When the cogwheel is made to turn, by turning the proximal rod of the drive member, the teeth of the cogwheel drive the first and the second bone screws forward simultaneously, by meshing with the helical indentation of the bone screw, through the first and the second bone screw passages of the bone fusion device, respectively, into a receiving bone thereby securing the bone fusion device in its intended place in a human or animal body. During the process, the guide member may move along the proximal rod of the drive member freely.

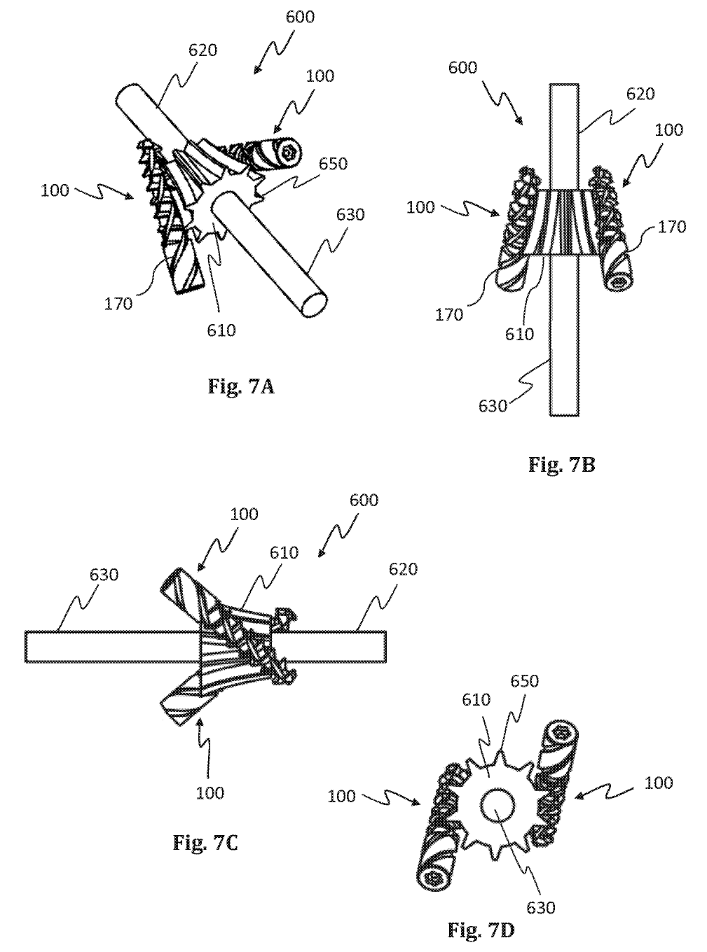

Turning now to the drawings, FIGS. 7A-7D illustrate an engagement of a drive member 600 of a fastening unit with the bone screws 100 to be inserted into a human or animal body, in accordance with an embodiment of the present disclosure. For the sake of simplicity and better illustration, neither a guiding member of the fastening unit nor a bone fusion device to be fixed by the bone screws is shown. The drive member 600 includes a concavely conical cogwheel 610 stably disposed between a distal portion 620 and a proximal portion 630 of a rod arrangement. The cogwheel 610 has radially projecting teeth 650 configured to mesh with a helical indentation 170 of the bone screw 100.

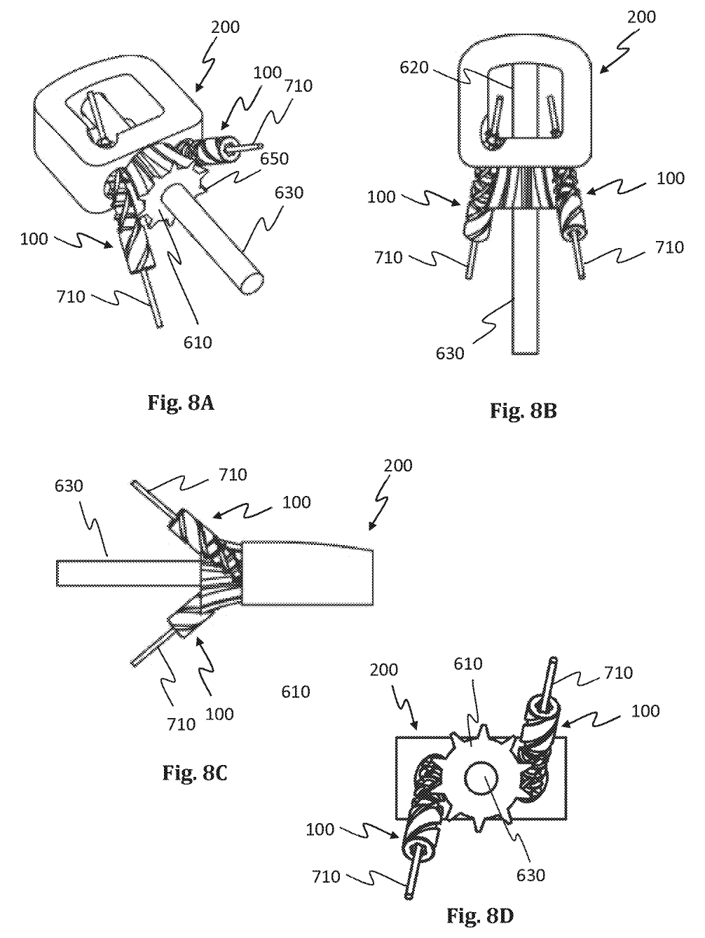

FIGS. 8A-8D illustrate the engagement shown in FIGS. 7A-7D, respectively, now completed with a bone fusion device 200 and guidewires 710 received in longitudinal bores of bone screws 100. For the sake of simple illustration, the whole fastening unit configured for fixing the bone fusion device 200 in its intended place in the body is not shown.

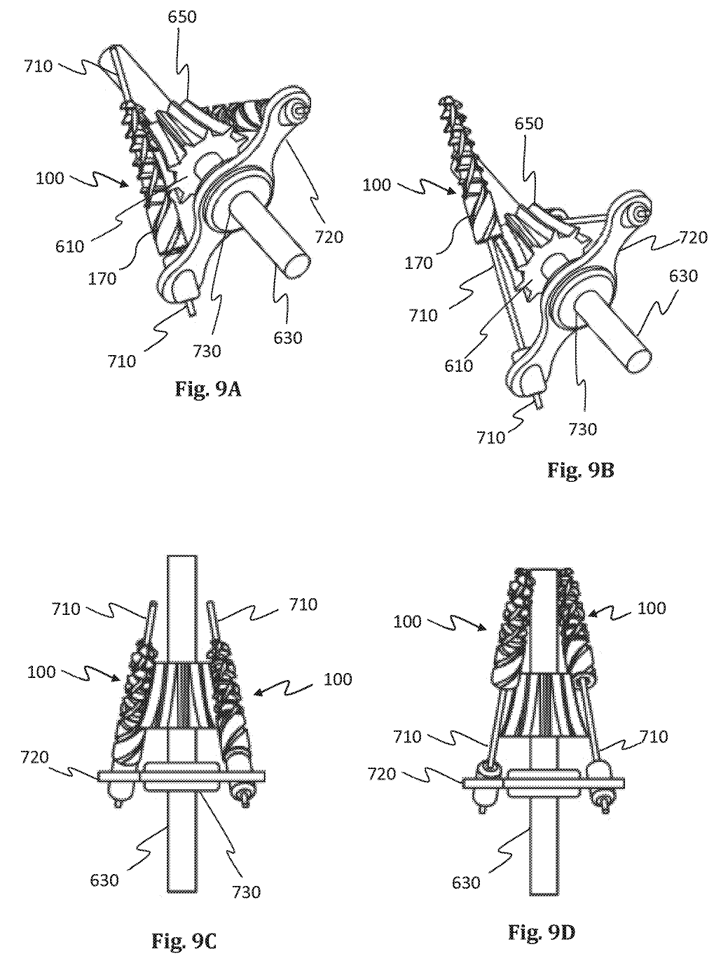

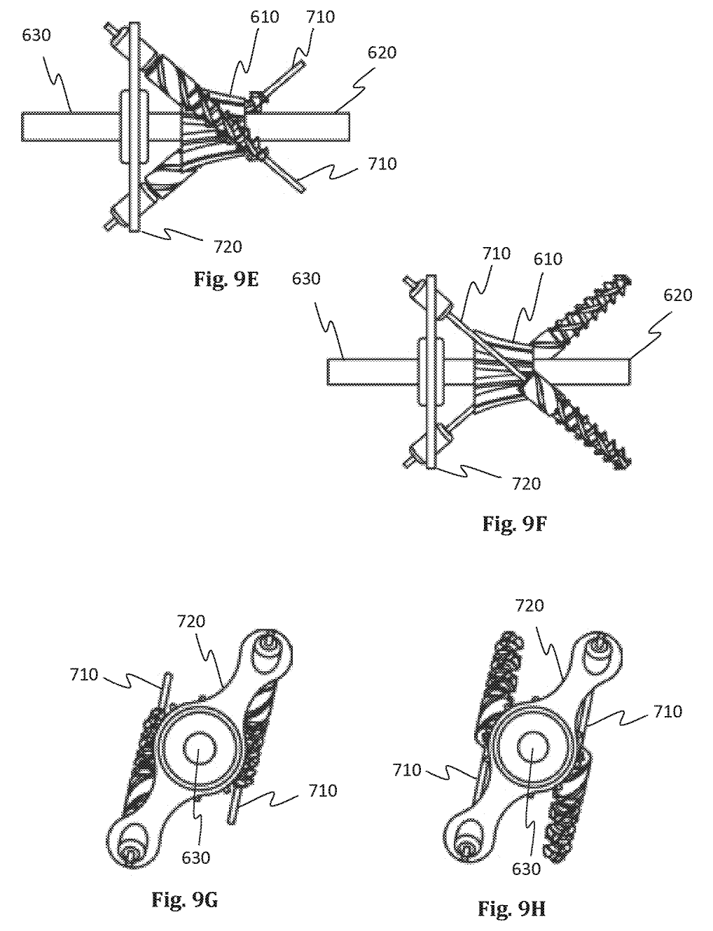

FIGS. 9A, 9C, 9E and 9G illustrate an engagement of two bone screws 100 with a fastening unit at an early phase of a process of fixing a bone fusion device in its desired location in a human or animal body, whereas FIGS. 9B, 9D, 9F and 9H illustrate the same engagement at a later phase of the fixing process, respectively, in accordance with an embodiment of the present disclosure. Rotating the proximal portion 630 of the rod arrangement results in rotation of the cogwheel 610 having radially projecting teeth 650 configured to mesh with a helical indentation 170 in bone screws 100, thereby driving the bone screws 100 forward. Each of the two guidewires 710 coupled from its one end to an elongated body 720 of the fastening unit is removably received in a longitudinal bore of the bone screw 100. The elongated body 720 has a central aperture 730 configured to receive a proximal portion 630 of the rod arrangement therethrough, such that the elongated body 720 is free to slide along said proximal portion 630 as the bone screws 100 are being driven forward by the cogwheel 610. Also the bone screws 100 are free to slide along the guidewires 710.

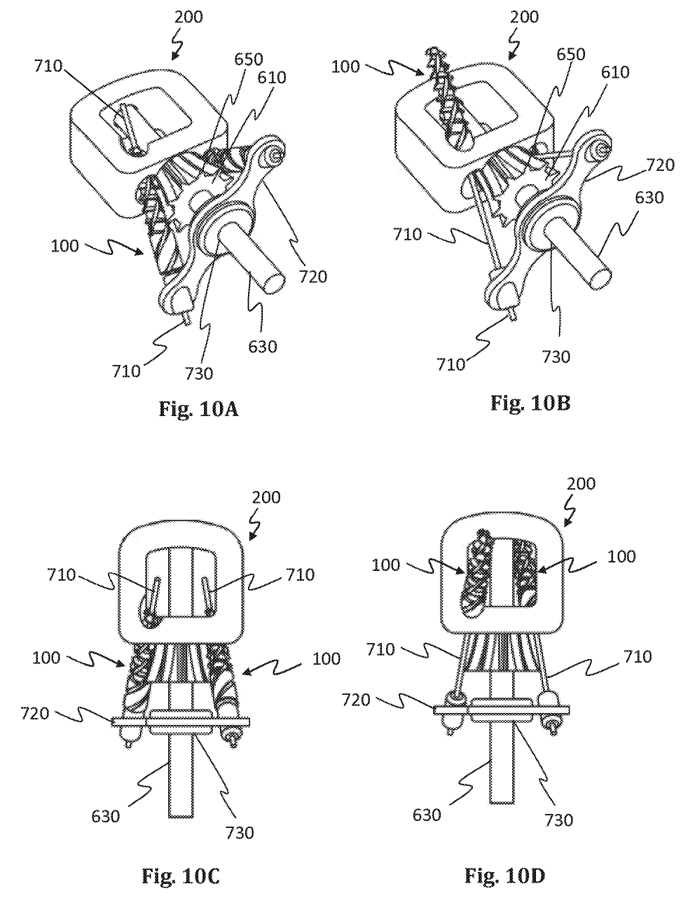

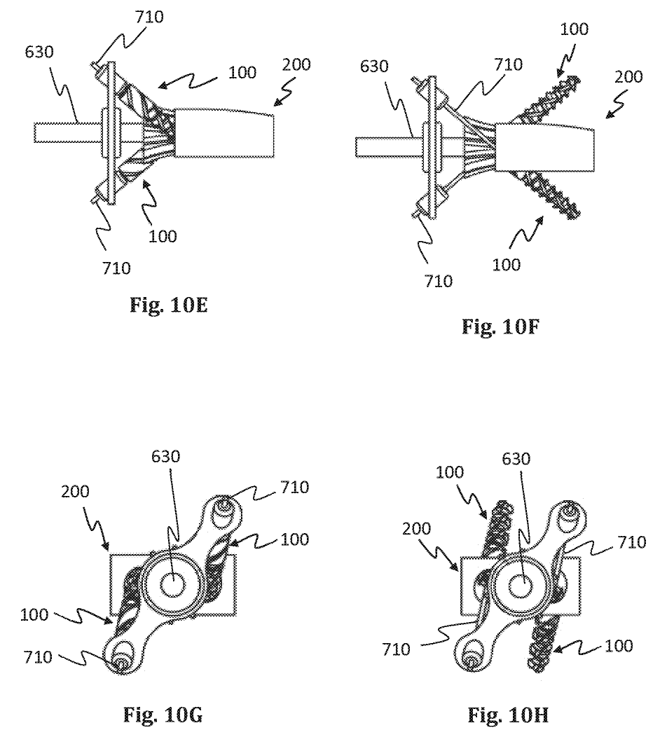

FIGS. 10A-10H correspond to FIGS. 9A-9H, respectively, but are now completed with a bone fusion device 200, in accordance with an embodiment of the present disclosure.

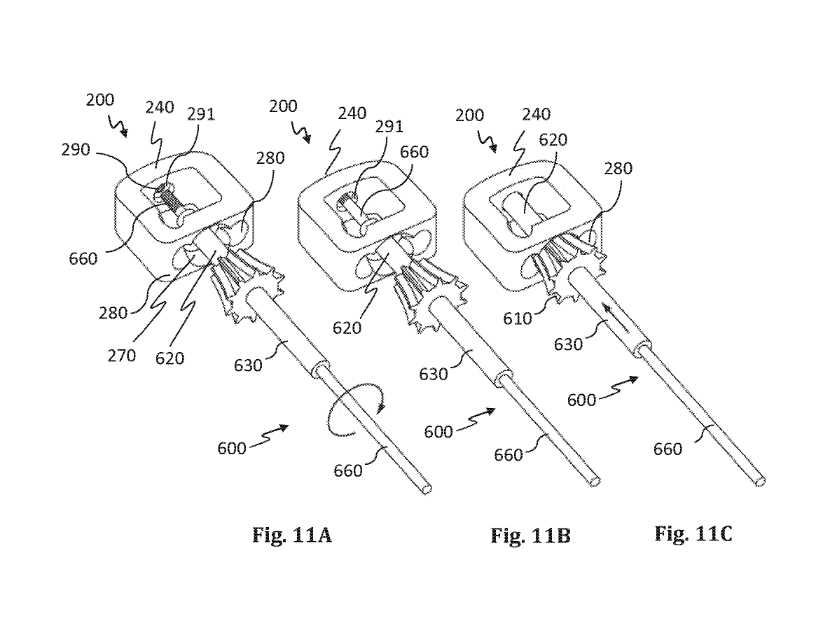

FIGS. 11A-11C represent a serial illustration of an engagement of a fastening unit with a bone fusion device shown in FIG. 3A at successive points in time, in accordance with an embodiment of the present disclosure. To be more specific, FIGS. 11A-11C show a drive member 600 of a fastening unit with a bone fusion device 200 in accordance with an embodiment of the present disclosure, wherein the drive member 600 is equipped with a fastening stick 660. For the sake of simplicity, neither a complete fastening unit nor bone screws to be inserted through bone screw passages 280 are shown. As illustrated in FIGS. 11A and 11B, the distal portion of the drive member 600, including the distal portion 620 of the rod arrangement and the distal part of the fastening stick 660 is first set through the access port 270, such that the distal end of the fastening stick 660 is received in an inner blind-hole 290 at the proximal surface of the posterior wall 240 of the bone fusion device 200. The drive member 600 is then removably fastened to the bone fusion device 200 by rotating the proximal portion of the fastening stick 660 such that the external thread at the distal end of the fastening stick 660 is screwed into the inner blind-hole 290 having an internal treading at the center of the outer blind hole 291. Once fastened, the rod arrangement including the cogwheel 610 is then pushed along the fastening stick 660 such that the distal end of the rod arrangement is received in an outer, non-threaded, blind-hole 291 of the bone fusion device 200 as illustrated in FIG. 11C. The cogwheel may then be made to rotate by turning the proximal portion 630 of the rod arrangement.

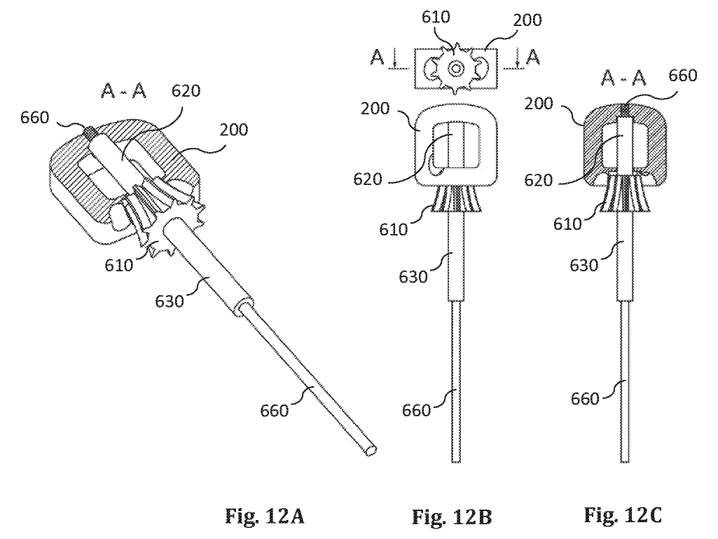

FIGS. 12A-12C show different views of the engagement shown in FIG. 11C.

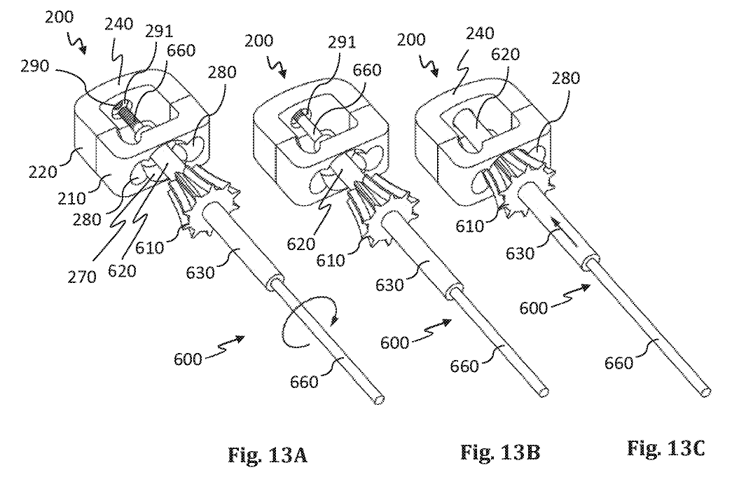

FIGS. 13A-13C represent a serial illustration of engagement of a drive member 600 of a fastening unit with a bone fusion device 200 in accordance with another embodiment of the present disclosure. Also in this embodiment, the drive member 600 is equipped with a fastening stick 660. As compared to the embodiment illustrated in FIGS. 11A-11C, the bone fusion device 200 is assembled from a plate member 210 and a spacer member 220. In other respects, the embodiment illustrated in FIGS. 13A-13C correspond to that illustrated in FIGS. 11A-11C.

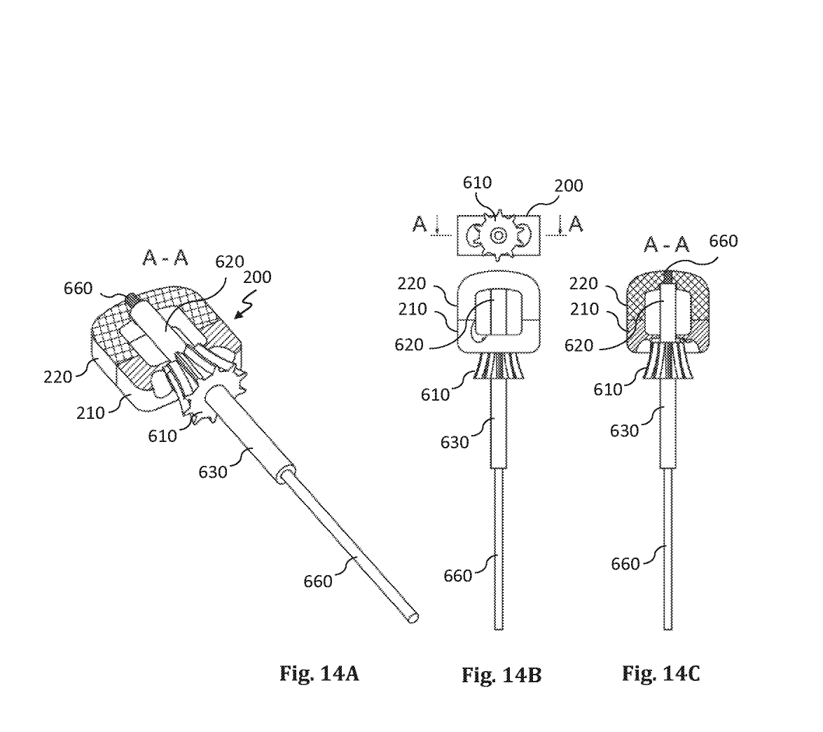

FIGS. 14A-14C show different views of the engagement shown in FIG. 13C.

1. A bone fusion system, comprising:
a bone fusion device having an anterior wall, posterior wall, a first lateral wall, a second lateral wall, and a central cavity open to a superior and an inferior surface, wherein
the anterior wall has a central access port extending therethrough, and at least one pair of a first bone screw passage and second bone screw passage in an oblique angle around the access port through the anterior wall,
the posterior wall having a blind-hole at the midpoint of its proximal surface, the blind-hole being configured to detachably receive a distal end of a rod arrangement in a fastening unit specifically configured for fixing the bone fusion device into a human or animal body; and
a bone screw in at least one of the bone screw passages, wherein the bone screw comprises:
an elongated shaft having a straight proximal region and a tapered distal region with one or more threads disposed along the length of the tapered distal region, and one or more helical indentations disposed along the elongated shaft in a direction opposite to the direction of said one or more threads, the one or more helical indentations being configured to engage the fastening unit such that bone screws in each of the first and second bone screw passages are driven simultaneously;
a head disposed at the proximal end of the elongated shaft; and
a tip disposed at the distal end of the elongated shaft.

2. The bone fusion system according to claim 1, wherein the bone fusion device is assembled from a plate member and a spacer member,
the plate member comprising:
an anterior wall, a first lateral extension at one end of the anterior wall and a second lateral extension at the other end of the anterior wall; and
the spacer member comprising:
a posterior wall, a first lateral wall at one end of the posterior wall and a second lateral wall at the other end of the posterior wall.

3. The bone fusion system according to claim 2, wherein the first lateral extension has an attachment portion configured to be coupled to a corresponding attachment portion in the first lateral wall, and the second lateral extension has an attachment portion configured to be coupled to a corresponding attachment portion in the second lateral wall.

4. The bone fusion system according to claim 1, wherein the first bone screw passage is angled through the anterior wall in an oblique upward direction and the second bone screw passage is angled through the anterior wall in an oblique downward direction.

5. The bone fusion system according to claim 4, wherein each of the bone screw passages also angled in an inward direction towards a vertical midline of the anterior wall along the anterior-posterior axis.

6. The bone fusion system according to claim 1, wherein the one or more helical indentations of the bone screw are disposed along the whole length of the elongated shaft.

7. The bone fusion system according to claim 1, wherein the bone screw further comprises: a longitudinal bore extending through said head, said straight proximal region, and at least a proximal portion of said tapered distal region.

8. The bone fusion system according to claim 7. wherein said longitudinal bore is configured to receive a guidewire of a fastening unit.

9. The bone fusion system according to claim 7, wherein the longitudinal bore is a longitudinal through-bore extending through the whole screw including the head, the elongated shaft and the tip.

10. The bone fusion system according to claim 7, wherein the shaft of the bone screw comprises one or more through-holes extending from said longitudinal bore through a lateral surface of the shaft, said one or more through-holes being disposed along at least a portion of the length of the shaft.

11. The bone fusion system according to claim 1, wherein said tip of the bone screw is a self-drilling tip.

12. The bone fusion system according to claim 1, wherein said head of the bone screw comprises a tooling recess configured for engaging with a fastening tool.

13. The bone fusion system according to claim 1, wherein the one or more helical indentations being configured to operably correspond to dimensions of the fastening unit.
